(19) United States
(12) Patent Application Publication
(10) Pub. No.: US 2025/0303039 A1
(43) Pub. Date: Oct. 2, 2025

(54) DIALYSIS FLUID CONTAINERS

(71) Applicant: Mozarc Medical US LLC, Minneapolis, MN (US)

(72) Inventors: Niccolò Chelli, Rufina (IT); Marialuisa Caiazzo, Modena (IT)

(21) Appl. No.: 19/094,180

(22) Filed: Mar. 28, 2025

Related U.S. Application Data
(60) Provisional application No. 63/571,557, filed on Mar. 29, 2024.

(51) Int. Cl. A61M 1/16

(52) U.S. Cl. CPC A61M 1/1668; A61M 2205/0205; A61M 2205/0238

(57) ABSTRACT

In some examples, an article includes a housing configured to receive a fluid for dialysis. The housing defines an interior surface configured to contact the fluid when the fluid is received in the housing. The interior surface includes at least one antimicrobial region. The at least one antimicrobial region defines a microscale texture configured to resist adhesion of bacteria and resist formation of biofilm. The microscale texture includes a plurality of microscale protrusions extending from the interior surface. In some examples, a system includes a dialysis container including the article and a dialysis unit coupled to the dialysis container.

### CROSS-REFERENCE TO RELATED APPLICATIONS

The present application claims the benefit of U.S. Provisional Application Ser. No. 63/571,557, filed Mar. 29, 2024, which is incorporated herein by reference in entirety.

### TECHNICAL FIELD

The present disclosure generally relates to dialysis fluid containers.

### BACKGROUND

A dialysis fluid container may be used to store a fluid for dialysis, for example, water or an aqueous fluid. For example, the fluid may be used for peritoneal dialysis.

### SUMMARY

In general, the present disclosure describes dialysis fluid containers that are configured to resist microbial growth and biofilm formation. When a fluid for dialysis is stored for a period of time in a dialysis fluid container, the fluid may tend to exhibit microbial growth. For example, a population of bacteria may grow, and tend to accumulate or attach to a surface, for example, an interior surface of the dialysis fluid container. The bacteria or other microorganisms may promote the formation of biofilm on the interior surface. In examples described herein, an interior surface of an article configured to receive a fluid for dialysis includes at least one antimicrobial region. The at least one antimicrobial region includes (e.g., defines) a microscale texture, which may deter bacterial adhesion and biofilm formation. Without being bound by theory, the microscale texture may exhibit hydrophobicity or superhydrophobicity, resulting in an antimicrobial effect.

In some examples, an example article includes a housing configured to receive a fluid for dialysis. The housing defines an interior surface configured to contact the fluid when the fluid is received in the housing. The interior surface includes at least one antimicrobial region. The at least one antimicrobial region includes a microscale texture configured to resist adhesion of bacteria and resist formation of biofilm. The microscale texture includes a plurality of microscale protrusions extending from the interior surface.

In some examples, an example system includes a dialysis container including an article, and a dialysis unit coupled to the dialysis container. The article includes a housing configured to receive a fluid for dialysis. The housing defines an interior surface configured to contact the fluid when the fluid is received in the housing. The interior surface includes at least one antimicrobial region. The at least one antimicrobial region includes a microscale texture configured to resist adhesion of bacteria and resist formation of biofilm. The microscale texture includes a plurality of microscale protrusions extending from the interior surface.

In some examples, an example method includes forming a housing configured to receive a fluid for dialysis. The housing defines an interior surface configured to contact the fluid when the fluid is received in the housing. The interior surface includes at least one antimicrobial region. The at least one antimicrobial region includes a microscale texture configured to resist adhesion of bacteria and resist formation of biofilm. The microscale texture includes a plurality of microscale protrusions extending from the interior surface.

The details of one or more examples of the techniques of this disclosure are set forth in the accompanying drawings and the description below. Other features, objects, and advantages of the techniques will be apparent from the description and drawings, and from the claims.

### BRIEF DESCRIPTION OF DRAWINGS

FIG. 1A is a diagram illustrating a top view of an example article including a housing configured to receive a fluid for dialysis.

FIG. 1B is a diagram illustrating a magnified partial view of the article of FIG. 1A showing an antimicrobial region of the article.

FIG. 1C is a diagram illustrating a magnified partial cross-sectional view of the article of FIG. 1A showing the antimicrobial region, where the cross-section is taken along line A-A in FIG. 1B and in the y-z plane.

FIG. 1D is a diagram illustrating a magnified partial top view of the article of FIG. 1A showing a plurality of microscale protrusions in a uniform square grid.

FIG. 2A is a diagram illustrating a top view of an example microscale texture including a plurality of microscale protrusions including a pillar.

FIG. 2B is a diagram illustrating a cross-sectional view of the microscale texture of FIG. 2A, where the cross-section is taken along line B-B in FIG. 2A and in the y-z plane.

FIG. 3 is a diagram illustrating a side view of an example microscale texture including a plurality of microscale protrusions including interleaved pillars of different heights.

FIG. 4 is a diagram illustrating a top view of an example microscale texture including a plurality of microscale protrusions including a first cluster and a second cluster.

FIG. 5A is a diagram illustrating a cross-sectional view of an example microscale protrusion including a bulge.

FIG. 5B is a diagram illustrating a top view of the microscale protrusion of FIG. 5A.

FIG. 6 is a diagram illustrating a top view of an example microscale protrusion including a lenticular bulge.

FIG. 7 is a diagram illustrating a partial side view of an example microscale texture including a plurality of microscale protrusions including a first cluster and a second cluster.

FIG. 8A is a diagram illustrating a top view of an example microscale texture including a plurality of microscale protrusions including a riblet.

FIG. 8B is a diagram illustrating a cross-sectional view of the riblet of FIG. 8A, where the cross-section is taken along line L-L in FIG. 8A and in the x-z plane.

FIG. 9 is a diagram illustrating a top view of an example microscale texture including a plurality of microscale protrusions including a cruciform.

FIG. 10 is a diagram illustrating a top view of an example microscale texture including a plurality of microscale protrusions including a pillar and a polyhedron.

FIG. 11 is a diagram illustrating a top view of an example microscale texture including a plurality of microscale protrusions including a pillar and a star.

FIG. 12 is a diagram illustrating a top view of an example microscale texture including a plurality of microscale protrusions including a pillar, a star, and a polyhedron.

FIG. 13 is a diagram illustrating a top view of an example microscale texture including a plurality of microscale protrusions including a riblet, a first polyhedron, and a second polyhedron.

FIG. 14 is a schematic block diagram illustrating an example system including a dialysis container and a dialysis unit.

### DETAILED DESCRIPTION

The present disclosure generally relates to articles including microscale textures configured to resist microbial growth, e.g., for antibiofouling. In some examples, the article includes a housing for use in dialysis systems, e.g., a fluid container.

Peritoneal dialysis is a home therapy treatment that requires relatively high storage volumes for dialysate bags. To save space and reduce logistical impact, the treatment may be performed using fluid prepared at the point of use, for example, using concentrates and water from a water for injection (WFI) system. However, water or aqueous fluid used in such systems may exhibit microbial growth or biofilm formation, for example, in stagnant water. Recirculation may be used to prevent and/or reduce the growth of biofilm. However, when fluid passes through a container, benefits of recirculation may be reduced with increasing container volume, which may result in lower recirculation velocities. Thus, WFI production may require specialized reservoirs with a recirculation system. Chemical, thermal, or ultraviolet disinfection and descaling may also be used to resist bacterial growth, but recirculation and disinfection may contribute to complexity and power consumption of peritoneal dialysis.

In examples described, here, articles include antimicrobial regions defining microscale textures that may resist microbial growth and biofilm formation. For example, an antimicrobial region may include a plurality of microscale protrusions configured to resist microbial engrafting and biofilm formation. The antimicrobial region may thus be one or more of an antibiofouling region, an antibacterial region, or an antibiofilm region. The term “antibacterial” refers to specifically targeting and killing bacteria. The term “antimicrobial” refers to an inhibition of growth of a wide range of microorganisms, including bacteria, viruses, fungi, and parasites. The term “antibiofilm” refers to reduction or prevention of formation, or disruption of existing biofilm, which include communities of microorganisms adhering to surfaces and/or their secretions. The term “antibiofouling” refers to preventing the accumulation of microorganisms, plants, algae, and animals on surfaces, particularly in aquatic environments.

The plurality of microscale protrusions may exhibit an antimicrobial effect via hydrophobicity (e.g., superhydrophobicity). In some examples, an article includes a housing having any suitable shape and defining an interior surface configured to receive fluid, the interior surface including at least one antimicrobial region.

Articles including antimicrobial regions according to the present disclosure may provide a stable antimicrobial impact, reduce or eliminate the need for recirculation systems or chemical disinfection in dialysis systems, reduce a number of operations performed by a clinician or a patient, reduce or eliminate a need to handle chemicals, require relatively low water consumption, be environmentally-friendly, be compatible with any rigid material (e.g., rigid polymeric material), compatible with any container design, size, or volume, and can be manufactured by many techniques.

The plurality of microscale protrusions may include protrusions in a predetermined two-dimensional layout, and which may include ordered or repeating patterns. For example, the plurality of microscale protrusions may include one or more of pillars, riblets, bulges, papillae, grooves, cruciform, stars, polyhedrons, or other forms. The protrusions may be uniform or varied in one or more of size, shape, or distribution. In some examples, characteristic geometrical dimensions of the plurality of microscale protrusions are sized from 1 μm to 500 μm. For example, such dimensions may promote hydrophobicity, or otherwise resist microbial growth and biofilm formation. In some examples, microscale textures defined by the plurality of microscale protrusions may provide a relatively high contact angle, such as greater than or equal to 135°, which may limit bacterial engraftment by reducing the potential of interaction with bacterial membranes.

Articles including antimicrobial regions according to the present disclosure may be configured to resist or prevent one or more of bacterial engraftment, bacterial growth, or biofilm formation, or promote washability of surfaces from microbiological contaminants (e.g., having a relatively low adhesiveness to microorganisms).

In some examples, articles including antimicrobial regions may include at least one antimicrobial agent (e.g., in addition to a microscale texture). In some examples, a roughness of an antimicrobial region may be reduced to deter bacterial adhesion or biofilm formation. For example, a surface roughness (Ra) of an antimicrobial region may be 0.4 or less, 0.3 or less, 0.2 or less, or 0.1 or less. In some examples, articles including antimicrobial regions may include a high performance or a high strength polymer, for example, a copolyester.

Without being bound by theory, microbial organisms such as bacteria may tend to colonize recessed areas and avoid protruding areas. Thus, micropatterns with relatively wider protruded areas (e.g., relative to recessed areas) may work better than those with narrower protruded areas. Further, a pattern of microscale protrusions having a more regular repetition with a same feature repeating all over the area may not resist bacterial growth or film formation as well as a pattern that includes an irregular repetition or geometry. Therefore, in some examples, mixed patterns with different types of protrusions (e.g., differing in shape or size or some geometric aspect) may exhibit better antimicrobial activity relative to uniform patterns. Additionally, reducing height and spacing of microscale protrusions may resist the tendency of bacteria to intrude within recessed regions, and ultimately promote antimicrobial activity.

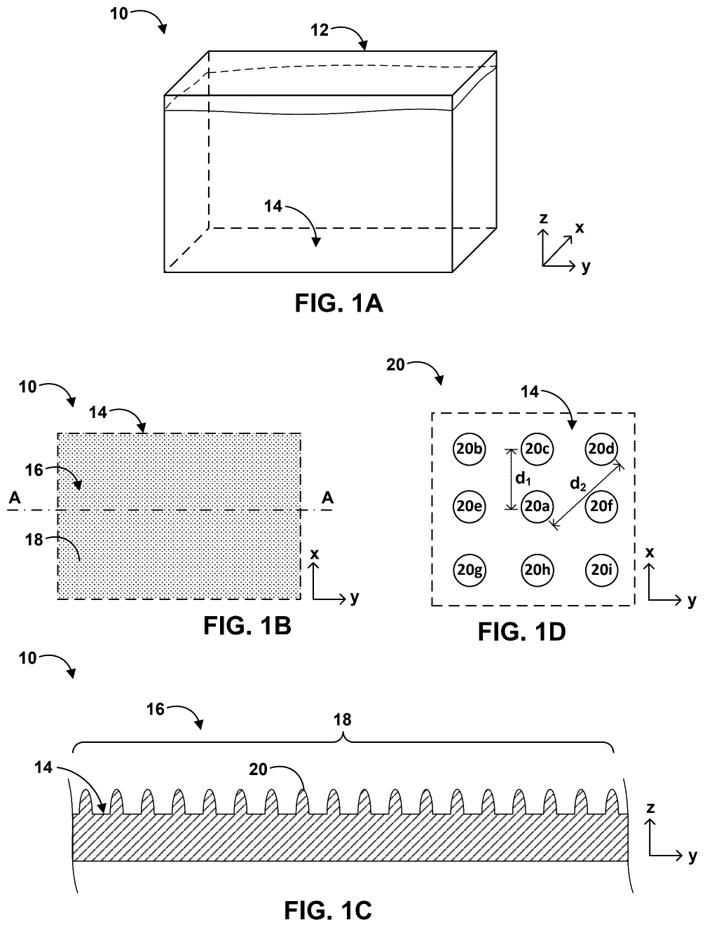

FIG. 1A is a diagram illustrating a top view of an example article 10 including a housing 12 configured to receive a fluid for dialysis. For example, the fluid for dialysis may include a fluid for peritoneal dialysis, hemodialysis, or other biomedical treatments that use a water-based solution storage between production and consumption, or a fluid in a water purification system. The fluid for dialysis may include water or an aqueous composition. Housing 12 may have any suitable shape or size. For example, housing 12 may define one or more of a planar face, a contoured or curved face, a linear edge, or a curved edge. In some examples, housing 12 is rounded. For example, corners or edges may be absent from a portion or an entirety of housing 12. In some examples, housing 12 is cuboidal or cylindrical. Housing 12 includes an opening through which a fluid can be received in a volume defined by an interior of housing 12. For example, housing 12 may include a base and a cover configured to be removably secured to the base (not shown), and the cover may be removable to allow the base to receive fluid. The cover may be configured to be fluidically and/or hermetically sealed to the base. Instead of or in addition to the cover and the base configuration, housing 12 may include a body that defines at least one opening (e.g., a port) through which fluid may be received. In some examples, housing 12 is configured to be moved or reoriented relative to a part of a dialysis system that includes a peritoneal dialysis cycler.

In some such examples, the body of housing 12 may be integral (e.g., unitary). In some examples, housing 12 is semi-rigid or substantially rigid (e.g., rigid or nearly rigid), for example, self-supporting or structured. Housing 12 may include any suitable composition, for example, a polymer or a glass. The composition of housing 12 may be biocompatible or sterilizable. In some examples, housing 12 includes, consists of, or consists essentially of a polymer. The polymer may include, for example, at least one of a polypropylene, a polycarbonate, a polyolefin (e.g., a polyethylene), a thermoplastic elastomer, a polyethylene terephthalate, a polyurethane, a cyclic olefin copolymer, a copolyester, a polysulfone, a polyphthalamide, a poly (phenylene methylene), or a silicone (e.g., polydimethylsiloxane). The copolyester may include a copolymer made from dimethyl terephthalate (DMT), cyclohexanedimethanol (CHDM), and 2,2,4,4-Tetramethyl-1,3-cyclobutanediol (CBDO) monomers.

Without being bound by theory, copolyesters may exhibit better performance for forming housing 12, compared to other types of polymers, for example, compared to cyclic olefin copolymers. For example, copolyesters may exhibit a relatively higher hydrophobicity or lower compatibility with extracellular matrix than certain other polymers, which may deter microbial attachment, colonization, or growth. Thus, copolyesters may be effective in resisting bacteria engraftment and biofilm growth (e.g., independent of texturing or without surface roughness). In some examples housing 12 (e.g., a matrix of housing 12) may be substantially formed of a copolyester (except for minor impurities and additives such as antimicrobial agent). In some examples, housing 12 may include a composite material, for example, a substrate polymer coated with a copolyester coating or co-molded, co-laminated, or co-extruded with the copolyester.

The polymer may include one or more additive, filler, or reinforcing material, for example, fibers (e.g., glass fibers). At least a portion of housing 12 may be transparent or translucent, for example, to determine a level of fluid in housing 12, or to detect indications of bacterial growth or biofilm formation. In some examples, an entirety of housing 12 is transparent or translucent. In some examples, article 10 includes a sensor configured to detect a level of fluid in housing 12.

Housing 12 defines an interior surface 14 configured to contact the fluid when the fluid is received in housing 12. For example, interior surface 14 may include at least a portion of one or more of a bottom surface, a top surface, an inclined surface, or a vertical surface with respect to gravity (which corresponds to the z-axis direction in the figures, orthogonal x-y-z axes being shown in the figures for ease of description). In some examples, interior surface 14 extends along x-y plane, for example, as a bottom surface with respect to gravity. In other examples, interior surface 14 extends along other planes (y-z plane, z-x plane, or some other plane). In some examples, interior surface 14 extends along a curved surface, and the orthogonal x-y-z axes are local axes with reference to a point of origin along the curved surface. Interior surface 14 may include any surface that may potentially contact the fluid. If the fluid is stored in housing 12 for a period of time, the fluid may exhibit microbial growth and/or biofilm formation. The microbial growth may include bacterial growth. While a portion of a microbial population may be suspended in a bulk of the fluid, another portion may tend to accumulate or contact interior surface 14, and grow on or alone interior surface 14. For example, a bacterial colony may form and grow along interior surface 14. In some examples, interior surface 14 may be susceptible to biofilm formation.

FIG. 1B is a diagram illustrating a magnified partial view of article 10 of FIG. 1A showing at least one antimicrobial region 16 of article 10. FIG. 1C is a diagram illustrating a magnified partial cross-sectional view of article 10 of FIG. 1A showing antimicrobial region 16, where the cross-section is taken along line A-A in FIG. 1B and in the y-z plane. For example, in some examples, interior surface 14 includes antimicrobial region 16. A portion of or an entirety of interior surface 14 may include antimicrobial region 16. In some examples, substantially an entirety of housing 12 (e.g., all of interior surface 14 except for certain parts, such as parts intended to engage with or mate with a cover) includes antimicrobial region 16 along interior surface 14.

Antimicrobial region 16 is configured to resist or prevent one or more of microbial contamination, microbial growth, or biofilm formation. Thus, antimicrobial region 16 may be configured to be an antibacterial region, an antibiofilm region, or an antibiofouling region. Antimicrobial region 16 defines a microscale texture 18 configured to resist or prevent one or more of adhesion of bacteria, growth of bacterial colonies, or formation of biofilm. Microscale texture 18 includes a plurality of microscale protrusions 20 extending from interior surface 14. One or more of the size, shape, and spacing of the plurality of microscale protrusions 20 facilitate the resisting adhesion of bacteria and formation of biofilm by interior surface 14, for example, by contributing to hydrophobicity or superhydrophobicity of antimicrobial region 16. In some examples, antimicrobial region 16 includes a substantially uniform microscale texture 18, for example, microscale texture 18 having a similar pattern of the plurality of microscale protrusions 20. In other examples, antimicrobial region 16 includes a plurality of microscale textures 18 including different patterns of microscale protrusions 20. For example, the patterns may differ in at least one of size, shape, distribution, or spacing of the plurality of microscale protrusions 20. In some examples, the plurality of microscale protrusions 20 is semi-rigid or substantially rigid (e.g., rigid or nearly rigid), for example, self-supporting.

The plurality of microscale protrusions 20 may include any suitable composition. In some examples, the plurality of microscale protrusions 20 includes, consists of, or consists essentially of at least one polymer. For example, the polymer may include any polymer described with reference to housing 12. In some examples, each microscale protrusion of the plurality of microscale protrusions 20 includes at least one polymer. In some such examples, each microscale protrusion of the plurality of microscale protrusions 20 consists of, or consists essentially of (e.g., except for impurities) at least one polymer. In some examples, a composition of housing 12 is the same as a composition of the plurality of microscale protrusions 20. In some such examples, at least one microscale protrusion of the plurality of microscale protrusions 20, or an entirety of the plurality of microscale protrusions 20, is integral with housing 12. For example, the plurality of microscale protrusions 20 may extend continuously from interior surface 14, without defining an interface. In other examples, at least one microscale protrusion of the plurality of microscale protrusions 20 is discrete from housing 12 and secured to housing 12, for example, by overmolding, coating, plastic welding, or adhesion processes. In some such examples, article 10 includes a liner defining plurality of microscale protrusions, and the liner may be inserted in housing 12 to define interior surface 14. In any of these examples, housing 12 may include a first composition (e.g., a first polymer or a glass), and plurality of microscale protrusions 20 may include a second composition (e.g., a second polymer).

In some examples, at least one microscale protrusion of the plurality of microscale protrusions 20 is solid, for example, having a solid interior absent of any voids. In other examples, at least one microscale protrusion of the plurality of microscale protrusions 20 defines a hollow interior.

The plurality of microscale protrusions 20 may have any size, shape, and spacing configured to resist adhesion of bacteria and formation of biofilm. For example, at least one of a length (e.g., in a direction along the x-axis), a width (e.g., in a direction along the y-axis), or a height (e.g., in a direction along the z-axis) of each microscale protrusion of the plurality of microscale protrusions 20 is less than or equal to 500 μm. In some examples, each of the length, the width, and the height of each microscale protrusion of the plurality of microscale protrusions 20 is less than or equal to 500 μm. In some examples, at least one of a length, a width, or a height of each microscale protrusion of the plurality of microscale protrusions 20 is less than or equal to 400 μm, 300 μm, 200 μm, 100 μm, 50 μm, 20 μm, or 10 μm. In some examples, at least one of a length, a width, or a height of each microscale protrusion of the plurality of microscale protrusions 20 is greater than or equal to 1 μm, 2 μm, 5 μm, 10 μm, 50 μm, 100 μm, 200 μm, 300 μm, or 400 μm. In some examples, at least one of a length, a width, or a height of each microscale protrusion of the plurality of microscale protrusions 20 is in a range from 1 μm to 500um, from 10 μm to 500 μm, from 100 μm to 500 μm, from 200 μm to 500 μm, from 300 μm to 500 μm, from 400 μm to 500 μm, from 50 μm to 350 μm, from 10 μm to 400 μm, from 10 μm to 300 μm, from 10 μm to 200 μm, from 10 μm to 100, or from 10 μm to 50 μm.

The length, the width, or the height of each microscale protrusion of the plurality of microscale protrusions 20 for a given antimicrobial region 16 and/or a given housing 12 may be substantially uniform (e.g., uniform to the extent permitted by manufacturing tolerances) or vary within predetermined ranges. In some examples, a maximum diameter of each microscale protrusion of the plurality of microscale protrusions 20 is in a range of from 10 μm to 50 μm. The maximum diameter of a microscale protrusion is a maximum of a distance between any pair of points along a contour of the microscale protrusion in the x-y plane or a plane parallel to the x-y plane. In some examples, a contour radius of each microscale protrusion of the plurality of microscale protrusions 20 is in a range of from 5 μm to 20 μm. The contour radius of a microscale protrusion is an average radius of a maximum perimeter defined by the microscale protrusion in any plane parallel to the x-y plane.

Housing 12 may further include at least one antimicrobial agent to promote an antimicrobial effect (e.g., in addition to or to supplement that created by microscale texture 18. The at least one antimicrobial agent may be dispersed in a matrix of housing 12. For example, the at least one antimicrobial agent may be dispersed throughout a bulk of housing 12. In some examples, the at least one antimicrobial agent is dispersed in a matrix of microscale texture 18. For example, the at least one antimicrobial agent may be dispersed only in a matrix of the microscale texture 18, while being absent from a remaining portion of housing 12. In other examples, the at least one antimicrobial agent may be dispersed both in the matrix of the microscale texture 18 and that of housing 12. In some examples, an antimicrobial coating applied to at least a portion of housing 12 includes the at least one antimicrobial agent. For example, the at least one antimicrobial agent may be present in the antimicrobial coating instead of, or in addition, being added to bulk of housing 12. In some such examples, the antimicrobial coating is applied over at least a portion of at least one antimicrobial region 16. In some examples, the antimicrobial coating is applied over at an entirety of at least one antimicrobial region 16. In some examples, the antimicrobial coating is applied over at an entirety of a major surface of housing 12 (e.g., over an interior surface of housing 12). The at least one antimicrobial agent may include any suitable antimicrobial agent, for example, a natural or synthetic antimicrobial agent. In some examples, the at least one antimicrobial agent is configured to release one or both of silver ions or copper ions.

In examples in which housing 12 includes a polymeric matrix, the at least one antimicrobial agent may be added to the polymeric matrix in any suitable manner. In some examples, the at least antimicrobial agent is co-molded together with at least one polymer of housing 12. For example, the at least antimicrobial agent may be mixed with a polymeric powder or pellet, or some other material. In some examples, the at least antimicrobial agent is dispersed in a polymeric carrier, which may in turn be mixed or combined with the polymeric powder or pellet to ultimately form housing 12. In some examples, the at least antimicrobial agent is included into a grain of the polymeric pellet or powder of housing 12. Including the at least antimicrobial agent in the grain of the polymeric pellet or powder (e.g., to a polypropylene pellet) may facilitate a substantially homogenous distribution of the at least one antimicrobial agent through the matrix of housing 12.

FIG. 1D is a diagram illustrating a magnified partial top view of article 10 of FIG. 1A showing plurality of microscale protrusions 20 in a uniform square grid. The nearest neighbors of a microscale protrusion consist of all microscale protrusions that do not have any intervening microscale protrusion. For example, in FIG. 1D, the nearest neighbors of microscale protrusion 20a are microscale protrusions 20b-20i. In the uniform square grid of microscale protrusions 20a-20i shown in FIG. 1D, four neighbor microscale protrusions 20c, 20e, 20f, and 20h are each at a distance di (center to center) from microscale protrusion 20a, and four microscale protrusions 20b, 20d, 20g, and 20i are each at a distance d₂ (center to center) from microscale protrusion 20a, with d₁ being less than d₂. The minimum inter-protrusion spacing for microscale protrusion 20a is d₁, while the average inter-protrusion spacing is (d₁+d₂)/2. An average inter-protrusion spacing (with respect to nearest neighbors), or a minimum inter-protrusion spacing (with respect to nearest neighbors), of the plurality of microscale protrusions 20 may be substantially uniform (e.g., uniform to the extent permitted by manufacturing tolerances) or vary within a predetermined range. In some examples, an average inter-protrusion spacing, or a minimum inter-protrusion spacing, is less than or equal to 500 μm. In some examples, the average inter-protrusion spacing, or the minimum inter-protrusion spacing for each microscale protrusion of the plurality of microscale protrusions 20 is less than or equal to 500 μm. In some examples, the average inter-protrusion spacing, or the minimum inter-protrusion spacing, of the plurality of microscale protrusions 20 is less than or equal to 400 μm, 300 μm, 200 μm, 100 μm, 50 μm, 20 μm, or 10 μm. In some examples, the average inter-protrusion spacing, or the minimum inter-protrusion spacing, of the plurality of microscale protrusions 20 is greater than or equal to 1 μm, 2 μm, 5 μm, 10 μm, 50 μm, 100 μm, 200 μm, 300 μm, or 400 μm. In some examples, the average inter-protrusion spacing, or the minimum inter-protrusion spacing, of the plurality of microscale protrusions 20 is in a range from 1 μm to 500 μm, from 10 μm to 500 μm, from 100 μm to 500 μm, from 200 μm to 500 μm, from 300 μm to 500 μm, from 400 μm to 500 μm, from 10 μm to 400 μm, from 10 μm to 300 μm, from 10 μm to 200 μm, from 10 μm to 100, or from 10 μm to 50 μm.

In some examples, at least a first microscale protrusion of the plurality of microscale protrusions 20 differs from at least a second microscale protrusion of the plurality of microscale protrusions 20 in at least one of a length, a width, a height, a contour, or a cross-sectional area.

A microscale protrusion of the plurality of microscale protrusions 20 defines any suitable cross-sectional contour in a plane parallel to or normal to interior surface 14. Further, in some examples, the cross-sectional contour of a respective microscale protrusion varies along a plane parallel to or normal to interior surface 14. In some examples, a microscale protrusion of the plurality of microscale protrusions 20 is elongated along any axis (for example, having a length greater than a width or a height, or a height greater than a width or a length, or a width greater than a height or a length).

In some examples, each microscale protrusion of the plurality of microscale protrusions 20 defines a same height. Thus, the plurality of microscale protrusions 20 may have a substantially uniform height. In other examples, at least a first microscale protrusion of the plurality of microscale protrusions differs from at least a second microscale protrusion of the plurality of microscale protrusions 20 in height. In some examples, the plurality of microscale protrusions 20 includes a first subplurality of microscale protrusions having a first height and second subplurality of microscale protrusions having a second height different from the first height. In some such examples, the first subplurality of microscale protrusions is interleaved with the second subplurality of microscale protrusions.

In some examples, each microscale protrusion of the plurality of microscale protrusions 20 is identical to at least one other protrusion of the plurality of microscale protrusions 20 in at least one of size or shape. In some such examples, each microscale protrusion of the plurality of microscale protrusions 20 is identical to at least one other protrusion of the plurality of microscale protrusions 20 in both size and shape. In some such examples, each microscale protrusion of the plurality of microscale protrusions 20 is identical in both size and shape.

In some examples, the plurality of microscale protrusions 20 is equally spaced along antimicrobial region 16. In other examples, the spacing between respective protrusions of plurality of microscale protrusions 20 may vary. For example, at least a first pair of microscale protrusions of the plurality of microscale protrusions may define a first minimum inter-protrusion distance, and at least a second pair of microscale protrusions of the plurality of microscale protrusions may define a second minimum inter-protrusion distance different from the first minimum inter-protrusion distance.

In some examples, the plurality of microscale protrusions 20 includes a plurality of clusters. For example, each cluster of the plurality of clusters defines a respective intra-cluster minimum distance different from a respective minimum inter-cluster distance. In some such examples, the respective intra-cluster minimum distance is less than the respective minimum inter-cluster distance.

Any of the structural features of the plurality of microscale protrusions 20 and individual protrusions 20 described herein can be used alone or in combination.

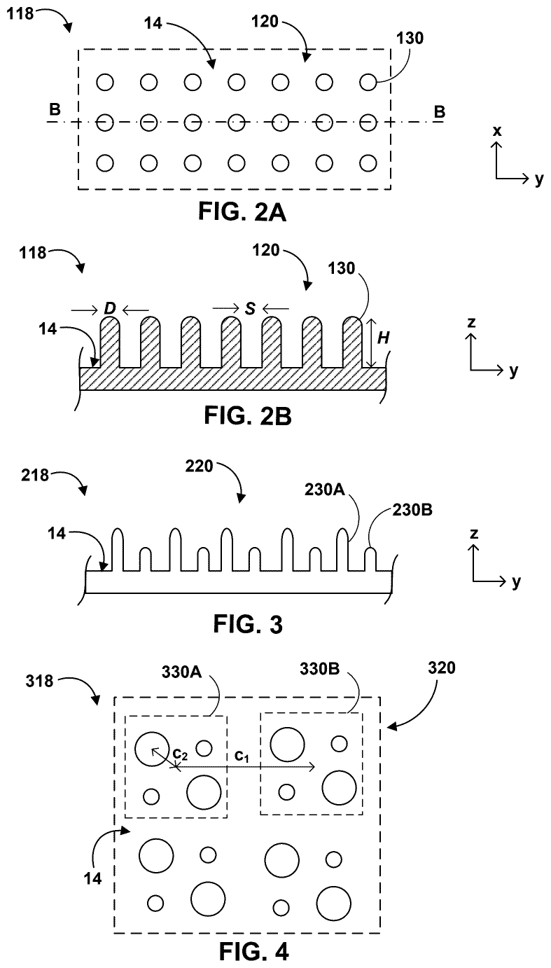

Protrusions 20 of the plurality of microscale protrusions 20 can include any suitable three-dimensional shape. In some examples, at least one microscale protrusion of the plurality of microscale protrusions 20 may include a pillar, a bulge, or a riblet, a cruciform, a star, or a polyhedron, as described with reference to FIGS. 2A to 13.

FIG. 2A is a diagram illustrating a top view of an example microscale texture 118 including a plurality of microscale protrusions 120 including a pillar 130. FIG. 2B is a diagram illustrating a cross-sectional view of microscale texture 118 of FIG. 2A, where the cross-section is taken along line B-B in FIG. A and in the y-z plane. Microscale texture 118 and the plurality of microscale protrusions 120 may be substantially similar to microscale texture 18 and the plurality of microscale protrusions 20 described with reference to FIGS. 1A to 1C in material and construction, but differ in certain geometric aspects. Pillar 130 may have a circular contour in the x-y plane or a plane parallel to the x-y plane, as shown in FIG. 2A. In other examples, pillar 130 may have an elliptical or polygonal contour.

Pillar 130 has any suitable dimensions for achieving an antimicrobial effect. Pillar 130 defines a diameter D, a height H, and a minimum inter-protrusion spacing S. In some examples, height H is greater than diameter D, such that pillar 130 is elongated along height H of pillar 130. In some examples, each protrusion of plurality of microscale protrusions 120 is a pillar, such that microscale protrusions 120 includes a plurality of pillars. Respective pillars of the plurality of pillars may be identical to pillar 130, for example, each pillar of the plurality of pillars having a same diameter D, a same height H, and a same minimum inter-protrusion spacing S. In other examples, at least one other pillar of the plurality of pillars may differ from pillar 130, in one or more of diameter D, height H, or minimum inter-protrusion spacing S. The diameter D, height H, or minimum inter-protrusion spacing S of respective pillars may be similar to that described with reference to microscale protrusions 20 of FIGS. 1A to 1C. In some examples, plurality of microscale protrusions 120 includes a pillar and at least one other form of microscale protrusion according to the present disclosure.

In the configuration shown in FIGS. 2A and 2B, the plurality of microscale protrusions 120 includes a plurality of pillars including pillar 130, arranged in a uniform square grid alone interior surface 14 of housing 12. Thus, the minimum inter-protrusion spacing S is the same as an average inter-protrusion spacing in such a configuration. However, in other examples, the grid may be non-uniform (for example, having a greater inter-protrusion spacing in rows compared to columns or vice versa), and the minimum inter-protrusion spacing S in those configurations may be less than the average inter-protrusion spacing. In still further examples, the plurality of pillars may be arranged in a hexagonal array, or any other suitable geometric spacing of pillars along microscale texture 118.

Pillar 130 may have a rounded end (e.g., a surface of pillar 130 furthest from interior surface 14 of housing 12), for example, as shown in FIG. 2B. The rounded end may have a circular, elliptical, or curved cross-section. In other examples, pillar 130 has an inclined, planar, conical, or flat end. In some examples, pillar 130 is cylindrical or conical.

FIG. 3 is a diagram illustrating a side view of an example microscale texture 218 including a plurality of microscale protrusions 220 including interleaved pillars of different heights. Microscale texture 218 and the plurality of microscale protrusions 220 may be substantially similar to microscale texture 118 and the plurality of microscale protrusions 120 described with reference to FIG. 2 in material and construction, but differ in certain geometric aspects. For example, plurality of microscale protrusions 220 may include a first plurality of pillars 230A having a first height, and a second plurality of pillars 230B having a second height different from the first height. The first plurality of pillars 230A may be interleaved with second plurality of pillars 230B in rows and/or columns, or in other geometric arrangements (e.g., rings or other cells). In some examples, first plurality of pillars 230A and second plurality of pillars 230B may respectively have a first diameter and a second diameter different from the first diameter. In other examples, first plurality of pillars 230A and second plurality of pillars 230B may differ in diameter, but have an identical height.

In some examples, microscale protrusions are clustered along interior surface 14 of housing 12, as described with reference to FIG. 4.

FIG. 4 is a diagram illustrating a top view of an example microscale texture 318 including a plurality of microscale protrusions 320 including a first cluster 330A and a second cluster 330B. Microscale texture 318 and the plurality of microscale protrusions 320 may be substantially similar to microscale texture 118 and the plurality of microscale protrusions 120 described with reference to FIGS. 2A and 2B in material and construction, but differ in certain geometric aspects. For example, first cluster 330A and second cluster 330B may each include microscale protrusions, and have an intra-cluster distance less than an inter-cluster distance. For example, the inter-cluster spacing is an average spacing c₁ between respective geometric centers of first cluster 330A and second cluster 330B, and the intra-cluster spacing of each cluster 330A and 330B is an average spacing c₂ between respective microscale protrusions of the respective cluster. In the example shown in FIG. 4, each cluster has an identical intra-cluster spacing and inter-cluster spacing. In other examples, at least one cluster may differ from at least one other cluster in one or both of intra-cluster spacing or inter-cluster spacing. Moreover, respective microscale protrusions of a cluster may be identical to or differ from other microscale protrusions within the cluster in one or more of height, diameter, or minimum inter-protrusion spacing.

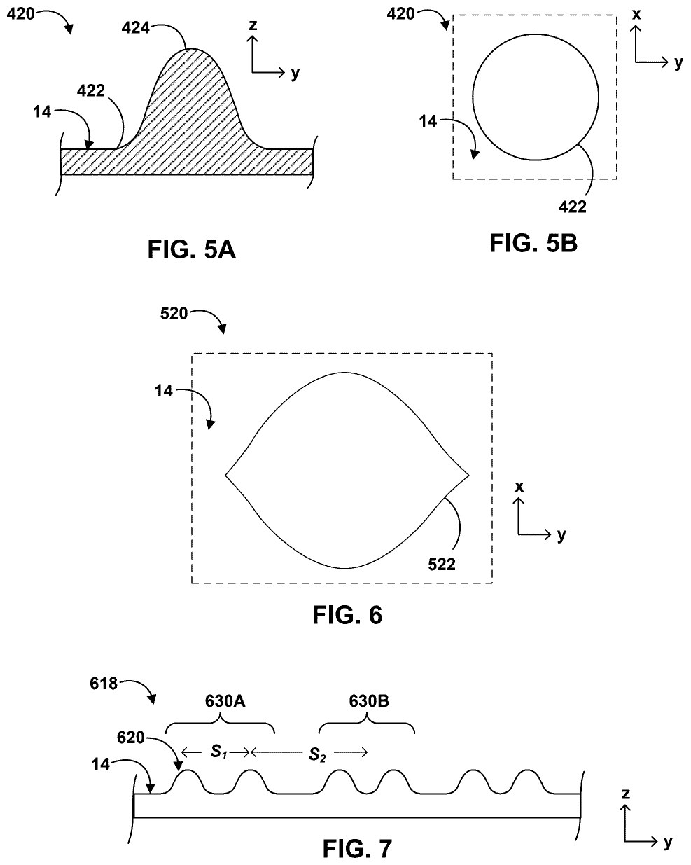

FIG. 5A is a diagram illustrating a cross-sectional view of an example microscale protrusion 420 including a bulge. FIG. 5B is a diagram illustrating a top view of the microscale protrusion 420 of FIG. 5A. A microscale texture (e.g., microscale textures 18, 118, 218, or 318) may include microscale protrusion 420. The bulge may extend from a base 422 (e.g., along interior surface 14) to a peak 424 (spaced from interior surface 14). In some examples, the bulge defines a gaussian contour, for example, in a cross-section from the base to the peak. Peak 422 may be a rounded peak, as shown in FIG. 5A, or may be a sharp peak. In some examples, the bulge extends to a single peak. In other examples, the bulge extends to multiple peaks. In some examples, base 422 defines a circular contour, as shown in FIG. 5B. In other examples, base 422 defines an elliptical or another curved contour.

In some examples, respective microscale protrusions of a plurality of microscale protrusions each includes a bulge. Thus, the plurality of microscale protrusions may include a plurality of bulges including bulge 420. The respective microscale protrusions may be identical or differ in one or more of height, diameter, or inter-protrusion spacing. For example, each bulge of the plurality of bulges defines a same maximum diameter. In other examples, the plurality of bulges may include a first subplurality of bulges defining a first diameter and a second subplurality of bulges defining a second diameter different from the first bugle diameter. In some examples, the plurality of bulges includes a plurality of bulge clusters (e.g., similar to the arrangement shown in FIG. 4), each plurality of bulge clusters including the first subplurality of bulges and the second subplurality of bulges. The respective microscale protrusions may vary in arrangement, spacing, grid, distribution, shape, or size, in a manner as described with reference to any other plurality of microscale protrusions according to the present disclosure, for example, the plurality of microscale protrusions 20, 120, 220, or 320.

FIG. 6 is a diagram illustrating a top view of an example microscale protrusion 520 including a lenticular bulge. Microscale protrusion 520 is similar to microscale protrusion 420 described with reference to FIGS. 5A and 5B, but differs in the contour. In particular, the lenticular bulge of microscale protrusion 520 has a base 522 defining a lenticular contour. The cross-section of microscale protrusion 520 may be similar to that of microscale protrusion 420 shown in FIG. 5A, or may have a circular or elliptical cross-section extending from base 522 to a peak of microscale protrusion 520.

In some examples, pillars or bulges may have a diameter in a range from 1 μm to 100 μm, from 1 μm to 50 μm, from 10 μm to 100 μm, or from 10 μm to 50 μm.

FIG. 7 is a diagram illustrating a cross-sectional view of an example microscale texture 618 including a plurality of microscale protrusions 620 including a first cluster 630A and a second cluster 630B. Microscale texture 618 and the plurality of microscale protrusions 620 may be substantially similar to microscale texture 118 and the plurality of microscale protrusions 120 described with reference to FIG. 2 or microscale protrusions 420 or 520 described with reference to FIGS. 5A or 6 in material and construction, but differ in certain geometric aspects. For example, each of first cluster 630A and second cluster 630B may define a same inter-protrusion spacing (or intra-cluster spacing) S₁ less than a uniform inter-cluster spacing S₂.

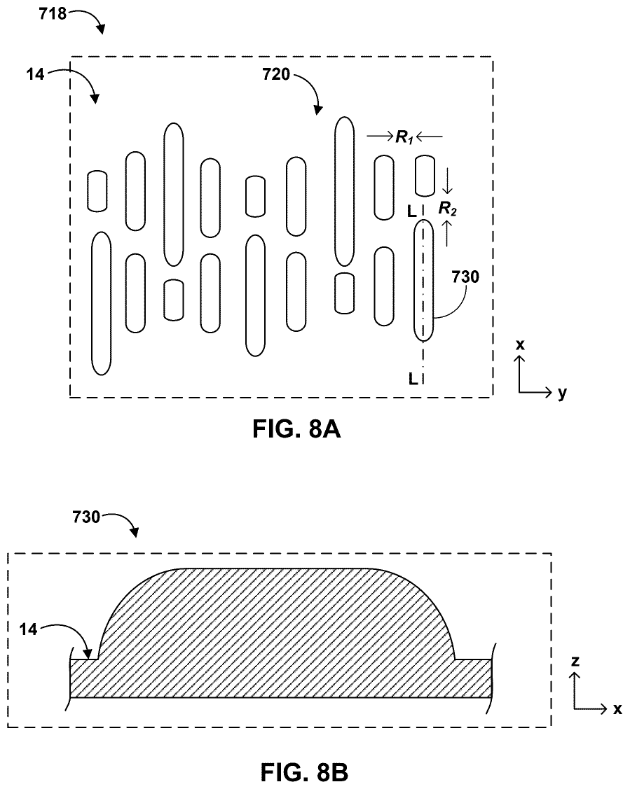

FIG. 8A is a diagram illustrating a top view of an example microscale texture 718 including a plurality of microscale protrusions 720 including a riblet 730. FIG. 8B is a diagram illustrating a cross-sectional view of riblet 730 along line L-L in FIG. 8A and in the x-z plane. Microscale texture 718 and the plurality of microscale protrusions 720 may be substantially similar to microscale texture 118 and the plurality of microscale protrusions 120 described with reference to FIGS. 2A and 2B in material and construction, but differ in certain geometric aspects. Riblet 730 defines a riblet length (along the x-axis direction, such as along line L-L) greater than a riblet width (alone the y-axis direction and transverse to line L-L).

The plurality of microscale protrusions 720 may include a plurality of riblets including riblet 730, and respective riblets of the plurality of riblets may be identical to or differ from riblet 730 in one or more of height, length, width, contour, a minimum lateral inter-riblet spacing (R₁), or a minimum axial inter-riblet spacing (R₂). While riblet 730 may extend from a base to a flat peak, as shown in FIG. 8B, in other examples, riblet 730 may define any suitable elongated, curved (e.g., elliptical) or polygonal shape in a cross-section between the base and the peak along line L-L. While riblet 730 may define a rounded riblet contour (e.g., in a plane along microscale texture 718) as shown in FIG. 8A, in other examples, riblet 730 may have a polygonal elongated contour.

In some examples, the plurality of microscale protrusions 720 includes a plurality of riblets including riblet 730, and the plurality of riblets includes a first subplurality of riblets defining a first riblet length (in a direction parallel to line L-L) and a second subplurality of riblets defining a second riblet length (in a direction parallel to line L-L) different from the first riblet length. In some such examples, the first subplurality of riblets is interleaved with the second plurality of riblets. In some examples, the plurality of microscale protrusions 720 includes a plurality of riblets having a distribution of riblet lengths, but having a similar riblet width, for example, as shown in FIG. 8A.

In some examples, the plurality of riblets have any suitable width, length or peak radius, for example, similar to dimensions described with reference to the plurality of microscale protrusions 20. In some examples, the plurality of riblets have a width in a range of from 50 μm to 350 μm. In some examples, the plurality of riblets have a length in a range of from 50 μm to 350 μm. In some examples, the plurality of riblets have a peak radius in a range of from 5 μm to 20 μm.

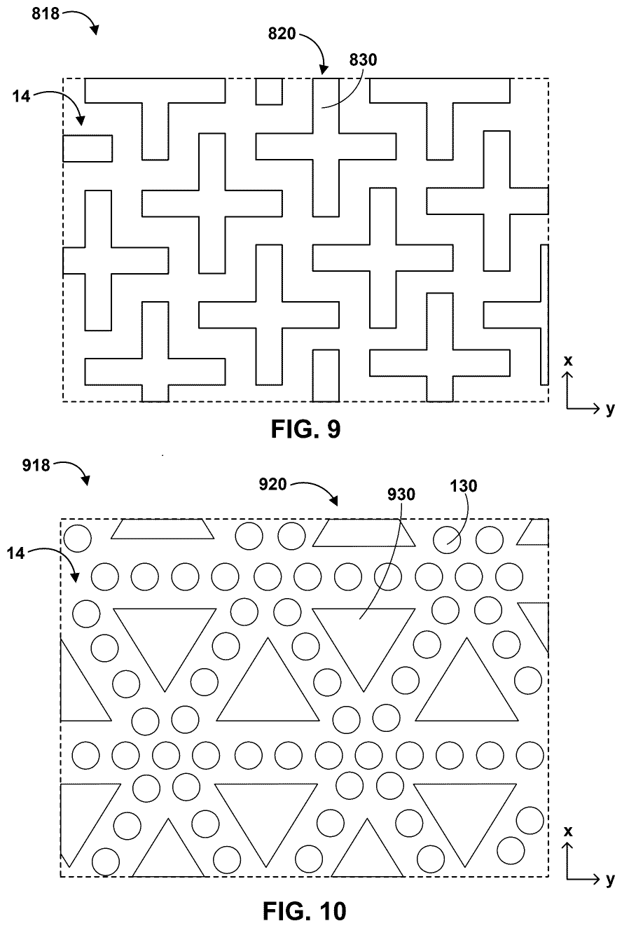

FIG. 9 is a diagram illustrating a top view of an example microscale texture 818 including a plurality of microscale protrusions 820 including a cruciform 830. Microscale texture 818 and the plurality of microscale protrusions 820 may be substantially similar to microscale texture 118 and the plurality of microscale protrusions 120 described with reference to FIGS. 2A and 2B in material and construction, but differ in certain geometric aspects. Cruciform 830 may define sharp or rounded edges or vertices. Cruciform 830 defines a major cruciform length (along the x-axis direction) and a major cruciform width (alone the y-axis direction). The major cruciform length may be the same as the major cruciform width (e.g., a cruciform that is symmetric about a geometric center of the cruciform) or may differ from the major cruciform width. Further, cruciform 830 includes four cruciform segments extending from a cruciform center. Each cruciform segment may be identical or differ in one or both of cruciform segment length (in a radial direction away from the cruciform center) or cruciform segment width (transverse to the radial direction).

In some examples, plurality of microscale protrusions 820 includes a plurality of cruciforms including cruciform 830. In some examples, plurality of microscale protrusions 820 consists of the plurality of cruciforms. In some examples, the plurality of cruciforms is interleaved with one another (e.g., both along the x-axis and the y-axis).

Each cruciform of the plurality of cruciforms may be identical in or differ in one or both of the major cruciform length and the major cruciform width. In some examples, each cruciform of the plurality of cruciforms is geometrically identical. One or both of the major cruciform length and the major cruciform width may be 100 μm or less, 90 μm or less, 80 μm or less, 70 μm or less, 60 μm or less, 50 μm or less, 40 μm or less, 30 μm or less, or 20 μm or less. One or both of the major cruciform length and the major cruciform width may be at least 20 μm, at least 30 μm, at least 40 μm, at least 50 μm, at least 60 μm, at least 70 μm, at least 80 μm, or at least 90 μm. In some examples, each of the major cruciform length and the major cruciform width is 23 μm.

The plurality of cruciforms may define an inter-cruciform spacing (e.g., between pairs of immediate neighboring cruciforms of the plurality of cruciforms). The inter-cruciform spacing may vary or be constant along one or both of the x-axis or along the y-axis. The inter-cruciform spacing along the x-axis be identical to or differ from the inter-cruciform spacing along the y-axis. In some examples, the inter-cruciform spacing along both the x-axis and the y-axis is constant and identical to each other. The inter-cruciform spacing may be 100 μm or less, 90 μm or less, 80 μm or less, 70 μm or less, 60 μm or less, 50 μm or less, 40 μm or less, 30 μm or less, 20 μm or less, 10 μm or less, or 5 μm or less. In some examples, the inter-cruciform spacing is at least 1 μm, at least 2 μm, at least 5 μm, at least 10 μm, at least 20 μm, at least 30 μm, at least 40 μm, at least 50 μm, at least 60 μm, at least 70 μm, at least 80 μm, or at least 90 μm. In some examples, the inter-cruciform spacing is 5 μm.

Cruciform 830 may define any suitable cruciform height. The cruciform height may be the same or different for each cruciform arm. In some examples, each cruciform arm of cruciform 830 has an identical cruciform height. The plurality of cruciforms may be identical in or differ in the cruciform height. In some examples, the cruciform height is 100 μm or less, 90 μm or less, 80 μm or less, 70 μm or less, 60 μm or less, 50 μm or less, 40 μm or less, 30 μm or less, or 20 μm or less. In some examples, the cruciform height is at least 10 μm, at least 20 μm, at least 30 μm, at least 40 μm, at least 50 μm, at least 60 μm, at least 70 μm, at least 80 μm, or at least 90 μm. In some examples, the cruciform height is 10 μm.

In some examples, both the major cruciform length and the major cruciform width for each cruciform is 23 μm, and each cruciform has a height of 10 μm and an inter-cruciform spacing of 5 μm.

FIG. 10 is a diagram illustrating a top view of an example microscale texture 918 including a plurality of microscale protrusions 920 including pillar 130 and a polyhedron 930. Microscale texture 918 and the plurality of microscale protrusions 920 may be substantially similar to microscale texture 118 and the plurality of microscale protrusions 120 described with reference to FIGS. 2A and 2B in material and construction, but differ in certain geometric aspects. Polyhedron 930 may define sharp or rounded edges or vertices. Polyhedron 930 may be regular (having identical faces) or irregular (different in size and relative orientation of faces). In some examples, polyhedron 930 defines at least one triangular face (e.g., an upper face may be triangular). In other examples, polyhedron 930 defines an upper face having at least four sides, at least five sides, at least six sides, or a greater number of sides. The other faces of polyhedron 930 may be rectangular or any other polygonal shape. In some examples, one face of polyhedron 930 is triangular, and four square or rectangular faces extend from the triangular face toward surface 14. Thus, polyhedron 930 may constitute a triangular pillar. The triangular face may define an equilateral triangle, an isosceles triangle, or a scalene triangle. The triangular face may have any suitable side length, for example, at least 2 μm, at least 5 μm, at least 10 μm, at least 20 μm, at least 30 μm, at least 40 μm, at least 50 μm, at least 60 μm, at least 70 μm, at least 80 μm, or at least 90 μm. In some examples, the side length is 100 μm or less, 90 μm or less, 80 μm or less, 70 μm or less, 60 μm or less, 50 μm or less, 40 μm or less, 30 μm or less, 20 μm or less, 10 μm or less, or 5 μm or less. In some examples, the side length is 10 μm.

In some examples, plurality of microscale protrusions 920 includes a plurality of polyhedrons including polyhedron 930. In some examples, plurality of microscale protrusions 920 further includes a plurality of pillars including pillar 130. In some examples, plurality of microscale protrusions 920 consists of the plurality of polyhedrons and the plurality of pillars. In some examples, the plurality of polyhedrons is interleaved with the plurality of pillars in any suitable pattern. For example, each polyhedron 930 may be laterally surrounded by one or more lateral layers or lines of pillars of the plurality of pillars. In some examples, each of the plurality of polyhedrons is geometrically identical and each of the plurality of pillars is geometrically identical. In some examples, the plurality of polyhedrons consists of triangular pillars. For example, the triangular pillars may point in the same direction (e.g., along the x-axis) or in opposite directions (e.g., staggered or alternating).

Plurality of microscale protrusions 920 may define an inter-protrusion spacing (e.g., between pairs of immediate neighboring protrusions of the plurality of protrusions). The inter-protrusions spacing may vary or be constant along one or both of the x-axis or along the y-axis. The inter-protrusions spacing may be identical to or differ for the plurality of pillars and the plurality of polyhedrons. In some examples, the inter-protrusion spacing is constant and identical to each other for both the plurality of pillars and the plurality of polyhedrons. The inter-protrusion spacing may be 100 μm or less, 90 μm or less, 80 μm or less, 70 μm or less, 60 μm or less, 50 μm or less, 40 μm or less, 30 μm or less, 20 μm or less, 10 μm or less, or 5 μm or less. In some examples, the inter-protrusion spacing is at least 1 μm, at least 2 μm, at least 5 μm, at least 10 μm, at least 20 μm, at least 30 μm, at least 40 μm, at least 50 μm, at least 60 μm, at least 70 μm, at least 80 μm, or at least 90 μm. In some examples, the inter-protrusion spacing is 2 μm.

Plurality of microscale protrusions 920 (e.g., one or both of the plurality of pillars or the plurality of polyhedrons) may define any suitable protrusion height. The protrusion height may be the same or different for the plurality of pillars or the plurality of polyhedrons. In some examples, each protrusion of plurality of microscale protrusions 920 has an identical protrusion height. In some examples, the protrusion height is 100 μm or less, 90 μm or less, 80 μm or less, 70 μm or less, 60 μm or less, 50 μm or less, 40 μm or less, 30 μm or less, or 20 μm or less. In some examples, the protrusion height is at least 5 μm, at least 10 μm, at least 20 μm, at least 30 μm, at least 40 μm, at least 50 μm, at least 60 μm, at least 70 μm, at least 80 μm, or at least 90 μm. In some examples, the protrusion height is 5 μm.

In some examples, plurality of microscale protrusions 920 consists of the plurality of pillars and the plurality of polyhedrons. In some such examples, the plurality of polyhedrons consists of identical triangle prism polyhedrons having a triangle side of 10 μm, the plurality of pillars consists of identical pillars of diameter 2 μm, and each protrusion has a height of 5 μm and an inter-protrusion spacing of 2 μm.

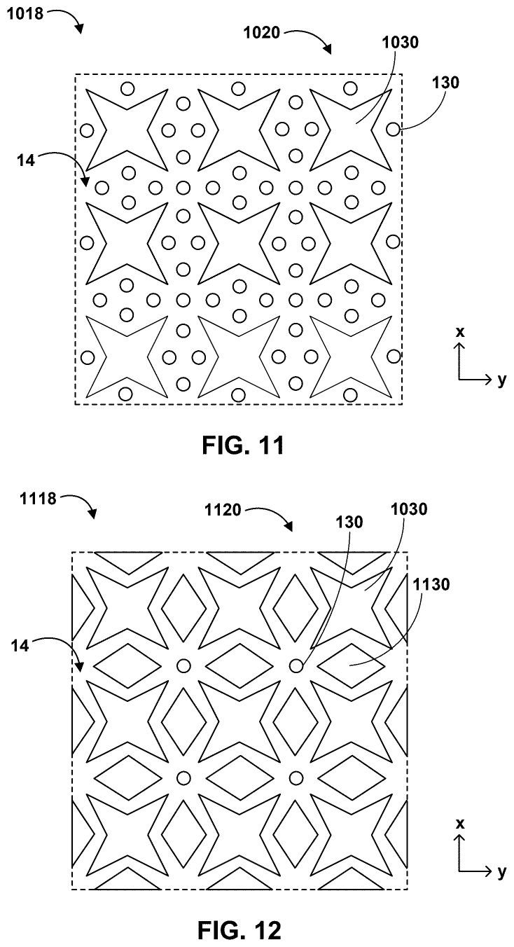

FIG. 11 is a diagram illustrating a top view of an example microscale texture 1018 including a plurality of microscale protrusions 1020 including pillar 130 and a star 1030.

Microscale texture 1018 and the plurality of microscale protrusions 1020 may be substantially similar to microscale texture 118 and the plurality of microscale protrusions 120 described with reference to FIGS. 2A and 2B in material and construction, but differ in certain geometric aspects. Star 1030 may define sharp or rounded edges or vertices. Star 1030 may be regular (having identical faces) or irregular (different in size and relative orientation of faces). In some examples, star 1030 is a polyhedron that defines at least one star-shaped face (e.g., an upper face may be star-shaped). The other faces of star 1030 may be rectangular or any other polygonal shape. In some examples, four square or rectangular faces extend from the star-shaped face toward surface 14. Thus, polyhedron 1030 may constitute a star-shaped pillar. The star-shaped face may be symmetric about one or both of x-axis or y-axis, and be uniform or varying in an arm length. For example, the star-shaped face may define four arms that are identical or different in arm length or arm width.

The star-shaped face may have any suitable dimensions. For example, a vertex-to-vertex spacing between a pair of neighboring vertices of the star-shaped face may be at least 2 μm, at least 5 μm, at least 10 μm, at least 20 μm, at least 30 μm, at least 40 μm, at least 50 μm, at least 60 μm, at least 70 μm, at least 80 μm, or at least 90 μm. In some examples, the vertex-to-vertex spacing is 100 μm or less, 90 μm or less, 80 μm or less, 70 μm or less, 60 μm or less, 50 μm or less, 40 μm or less, 30 μm or less, 20 μm or less, 10 μm or less, or 5 μm or less. In some examples, the vertex-to-vertex spacing is 10 μm.

In some examples, plurality of microscale protrusions 1020 includes a plurality of stars including star 1030. In some examples, plurality of microscale protrusions 1020 further includes a plurality of pillars including pillar 130. In some examples, plurality of microscale protrusions 1020 consists of the plurality of stars and the plurality of pillars. In some examples, the plurality of stars is interleaved with the plurality of pillars in any suitable pattern. For example, each star 1030 may be laterally surrounded by one or more lateral layers or lines of pillars of the plurality of pillars. In some examples, each of the plurality of stars is geometrically identical and each of the plurality of pillars is geometrically identical.

Plurality of microscale protrusions 1020 may define an inter-protrusion spacing (e.g., between pairs of immediate neighboring protrusions of the plurality of protrusions). The inter-protrusions spacing may vary or be constant along one or both of the x-axis or along the y-axis. The inter-protrusions spacing may be identical to or differ for the plurality of pillars and the plurality of stars. In some examples, the inter-protrusion spacing is constant and identical to each other for both the plurality of pillars and the plurality of stars. The inter-protrusion spacing may be 100 μm or less, 90 μm or less, 80 μm or less, 70 μm or less, 60 μm or less, 50 μm or less, 40 μm or less, 30 μm or less, 20 μm or less, 10 μm or less, or 5 μm or less. In some examples, the inter-protrusion spacing is at least 1 μm, at least 2 μm, at least 5 μm, at least 10 μm, at least 20 μm, at least 30 μm, at least 40 μm, at least 50 μm, at least 60 μm, at least 70 μm, at least 80 μm, or at least 90 μm. In some examples, the inter-protrusion spacing is 2 μm.

Plurality of microscale protrusions 1020 (e.g., one or both of the plurality of pillars or the plurality of stars) may define any suitable protrusion height. The protrusion height may be the same or different for the plurality of pillars or the plurality of stars. In some examples, each protrusion of the plurality of microscale protrusions has an identical protrusion height. In some examples, the protrusion height is 100 μm or less, 90 μm or less, 80 μm or less, 70 μm or less, 60 μm or less, 50 μm or less, 40 μm or less, 30 μm or less, or 20 μm or less. In some examples, the protrusion height is at least 5 μm, at least 10 μm, at least 20 μm, at least 30 μm, at least 40 μm, at least 50 μm, at least 60 μm, at least 70 μm, at least 80 μm, or at least 90 μm. In some examples, the protrusion height is 5 μm.

In some examples, plurality of microscale protrusions 1020 consists of the plurality of pillars and the plurality of stars. In some such examples, the plurality of stars consists of identical star-faced polyhedrons having a major diameter of 10 μm, the plurality of pillars consists of identical pillars of diameter 2 μm, and each protrusion has a height of 5 μm and an inter-protrusion spacing of 2 μm.

FIG. 12 is a diagram illustrating a top view of an example microscale texture 1118 including a plurality of microscale protrusions 1120 including pillar 130, star 1030, and a polyhedron 1130. Plurality of microscale protrusions 1120 is substantially similar to plurality of microscale protrusions 1020 described with reference to FIG. 11, but with certain pillars of the plurality of pillars including pillar 130 being replaced by polyhedron 1130. In some examples, polyhedron 1130 is a diamond-faced polyhedron (having a diamond-shaped face). Polyhedron 1130 may be symmetric about one or both of a major diamond axis and a minor diamond axis defined by a diamond-shaped face. The inter-protrusion spacing, protrusion height, and geometry of the plurality of pillars and the plurality of stars may be similar to that described with reference to FIG. 11. The diamond-faced polyhedron may have any suitable major diamond length and minor diamond length (major and minor diagonals). In some examples, one or both of the major diamond length or the minor diamond length may be in a similar range as that described with reference to star 1030. In some examples, the major diamond length is 10 μm and the minor diamond length is 6 μm.

In some examples, plurality of microscale protrusions 1120 consists of the plurality of pillars, the plurality of stars, and the plurality of polyhedrons. In some such examples, the plurality of stars consists of identical star-faced polyhedrons having a major diameter of 10 μm, the plurality of pillars consists of identical pillars of diameter 2 μm, the plurality of polyhedrons consists of identical diamond-faced polyhedrons of major length 10 μm and minor length μm, and each protrusion has a height of 5 μm and an inter-protrusion spacing of 2 μm.

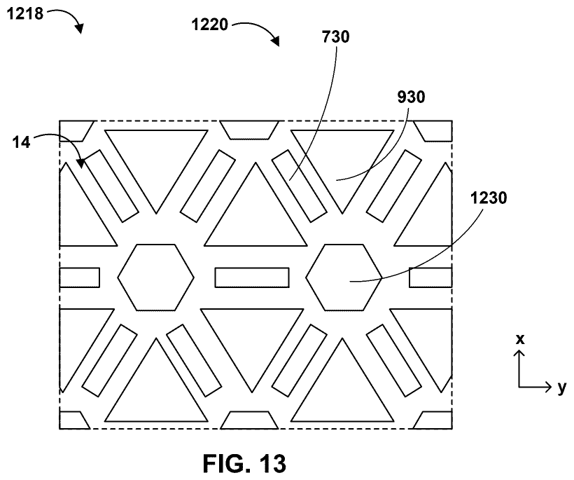

FIG. 13 is a diagram illustrating a top view of an example microscale texture 1218 including a plurality of microscale protrusions 1220 including riblet 730 (e.g., described with reference to FIGS. 8A and 8B), first polyhedron 930 (e.g., described with reference to FIG. 10), and a second polyhedron 1230. First polyhedron 930 may be a triangle-faced polyhedron. Polyhedron 1230 may define a regular or irregular polygonal face. In some examples, second polyhedron 1230 is a hexagonal-faced polyhedron (e.g., a hexagonal prism). In some examples, polyhedron 1230 is a regular hexagonal prism (e.g., having a regular hexagonal face). Each protrusion of plurality of microscale protrusions 1220 may define sharp or rounded edges or vertices.

The inter-protrusion spacing, protrusion height, and geometry of the plurality of pillars and the plurality of first polyhedrons may be similar to that described with reference to FIG. 12. A hexagonal face of second polyhedron 1230 may have any suitable side length, for example, at least 2 μm, at least 5 μm, or at least 10 μm. In some examples, the side length is 10 μm or less, 5 μm or less, or 2 μm or less. In some examples, the side length is 4 μm.

Riblet 730 may have any suitable dimensions. In some examples, riblet 730 has a width of 2 μm and a length of 8 μm.

In some examples, plurality of microscale protrusions 1020 includes a plurality of first polyhedrons including first polyhedron 930, plurality of second polyhedrons including second polyhedrons 1230, and a plurality of riblets including riblet 730. In some examples, plurality of microscale protrusions 1220 consists of the plurality of first polyhedrons, the plurality of second polyhedrons, and the plurality of riblets. In some examples, the plurality of first polyhedrons, the plurality of second polyhedrons, and the plurality of riblets are interleaved in any suitable pattern. For example, each second polyhedron 1230 may be laterally surrounded by one or more lateral layers or lines of first polyhedrons of the plurality of first polyhedrons or the riblets of the plurality of riblets. In some examples, each of the plurality of riblets is geometrically identical, each of the plurality of first polyhedrons is geometrically identical, and each of the plurality of second polyhedrons is geometrically identical.

While various geometrical aspects and arrangements have been described for example microscale textures or microscale protrusions with reference to FIGS. 1A to 13, one or more geometrical aspects and arrangements may be combined in any suitable manner.

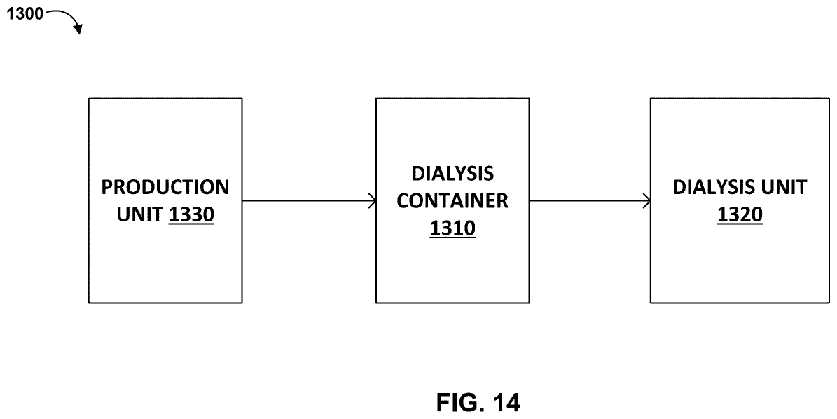

FIG. 14 is a schematic block diagram illustrating an example system 1300 including a dialysis container 1310 and a dialysis unit 1320. Dialysis container 1310 may include any article according to the present disclosure, for example, including any antimicrobial region, any microscale texture, or any plurality of microscale protrusions according to the present disclosure. Dialysis unit 1320 is configured to be fluidically coupled to a peritoneal cavity of a patient, for example, to perform peritoneal dialysis. However, system 1300 may be used to perform any dialysis procedure. For example, a fluid for dialysis may be transferred from dialysis container 1310 to dialysis unit, which may in turn supply the fluid to irrigate the peritoneal cavity of the patient. System 1300 may further include a production unit 1330 configured to supply or replenish fluid in dialysis container 1310.

Certain systems for dialysis, for example, peritoneal dialysis, may include a circulation unit or recirculation line to recirculate fluid for dialysis within the systems, for example, to resist microbial growth. Certain systems may include a disinfection source (for example, chemical disinfection, thermal disinfection, a heater, or ultraviolet disinfection), a chemical disinfection line coupled to one or more components of the system, to resist microbial growth. In examples according to the present disclosure, system 1300 does not include one or more of a circulation unit, a recirculation line, a disinfection source, or a disinfection line, and yet exhibits a comparable or better resistance to microbial growth and biofilm formation as systems including one or more such components. For example, system 1300 may not include a recirculation unit configured to recirculate fluid through the housing of the article. As another example, system 1300 may not include a disinfection source or a disinfection line configured to disinfect the interior surface of the housing.

Any suitable technique may be used to form articles, housings, microscale textures, microscale projections, or systems according to the present disclosure. In some examples, a method for forming an article includes fabricating the microscale texture by at least one of photolithography, electron beam lithography, soft lithography, x-ray lithography, plasma etching, reactive ion etching, chemical vapor deposition, layer-by-layer deposition, plasma-enhanced chemical vapor deposition, micro-molding, or additive manufacturing. In some examples, a housing of the article is integrally formed with the microscale texture. In other examples, the microscale texture is machined into or onto an interior surface of the housing, or deposited on the interior surface, for example, by masking, coating, stamping, overmolding, or additive manufacturing.

While articles, housings, microscale textures, or microscale protrusions according to the present disclosure may be used for dialysis, they may also be used for other applications, for example, hemodialysis, water purification processes, and water-for-injection (WFI) manufacturing, or any biomedical application requiring storage of water or aqueous solutions before or during production or consumption.

The following clauses describe examples according to the present disclosure.

Clause 1: An article including: a housing configured to receive a fluid for dialysis, where the housing defines an interior surface configured to contact the fluid when the fluid is received in the housing, where the interior surface includes at least one antimicrobial region, where the at least one antimicrobial region defines a microscale texture configured to resist adhesion of bacteria and resist formation of biofilm, and where the microscale texture includes a plurality of microscale protrusions extending from the interior surface.

Clause 2: The article of clause 1, where at least one microscale protrusion of the plurality of microscale protrusions is solid.

Clause 3: The article of any of clauses 1 or 2, where at least one microscale protrusion of the plurality of microscale protrusions defines a hollow interior.

Clause 4: The article of any of clauses 1 to 3, where at least one microscale protrusion of the plurality of microscale protrusions is integral with the housing.

Clause 5: The article of any of clauses 1 to 4, where at least one microscale protrusion of the plurality of microscale protrusions is discrete from the housing and secured to the housing.

Clause 6: The article of any of clauses 1 to 5, where the plurality of microscale protrusions includes at least one of a riblet, a pillar, a bulge, a cruciform, a star, or a polyhedron.

Clause 7: The article of clause 6, where the plurality of microscale protrusions includes the riblet, and where the riblet defines a riblet length greater than a riblet width.

Clause 8: The article of clause 7, where the riblet defines a rounded riblet contour.

Clause 9: The article of any of clauses 6 to 8, where the plurality of microscale protrusions includes a plurality of riblets including the riblet, and where the plurality of riblets includes a first subplurality of riblets defining a first riblet length and a second subplurality of riblets defining a second riblet length different from the first riblet length.

Clause 10: The article of clause 9, where the first subplurality of riblets is interleaved with the second plurality of riblets.

Clause 11: The article of any of clauses 6 to 10, where the plurality of microscale protrusions includes the pillar, and where the pillar defines a circular contour or an elliptical contour.

Clause 12: The article of any of clauses 6 to 11, where the plurality of microscale protrusions includes a plurality of pillars including the pillar, and where each pillar of the plurality of pillars has a same maximum diameter.

Clause 13: The article of any of clauses 6 to 12, where the plurality of microscale protrusions includes the bulge, and where the bulge defines a gaussian contour.

Clause 14: The article of any of clauses 6 to 13, where the plurality of microscale protrusions includes the bulge, and where the bulge extends to a single peak.

Clause 15: The article of any of clauses 6 to 14, where the plurality of microscale protrusions includes a plurality of bulges including the bulge.

Clause 16: The article of clause 15, where each bulge of the plurality of bulges defines a same maximum diameter.

Clause 17: The article of clause 16, where the plurality of bulges includes a first subplurality of bulges defining a first diameter and a second subplurality of bulges defining a second diameter different from the first bugle diameter.

Clause 18: The article of clause 17, where the plurality of bulges includes a plurality of bulge clusters, each plurality of bulge clusters including the first subplurality of bulges and the second subplurality of bulges.

Clause 19: The article of any of clauses 6 to 18, where the plurality of microscale protrusions includes a plurality of stars including the star, and where the plurality of stars is interleaved with the plurality of pillars.

Clause 20: The article of any of clauses 6 to 19, where the plurality of microscale protrusions includes a plurality of polyhedrons including the polyhedron, and wherein the plurality of polyhedrons is interleaved with the plurality of pillars and the plurality of stars.

Clause 21: The article of any of clauses 6 to 20, where the plurality of microscale protrusions includes a plurality of cruciforms including the cruciform, and where the plurality of cruciforms is interleaved with one another.

Clause 22: The article of clause 21, where each cruciform of the plurality of cruciforms has a height of 20 μm, a width of 23 μm, and an inter-cruciform spacing of 5 μm.

Clause 23: The article of any of clauses 1 to 22, where at least a first microscale protrusion of the plurality of microscale protrusions differs from at least a second microscale protrusion of the plurality of microscale protrusions in at least one of a length, a width, a height, a contour, or a cross-sectional area.

Clause 24: The article of any of clauses 1 to 23, where each microscale protrusion of the plurality of microscale protrusions defines a same height.

Clause 25: The article of any of clauses 1 to 24, where at least a first microscale protrusion of the plurality of microscale protrusions differs from at least a second microscale protrusion of the plurality of microscale protrusions in height.

Clause 26: The article of any of clauses 1 to 25, where the plurality of microscale protrusions includes a first subplurality of microscale protrusions having a first height and second subplurality of microscale protrusions having a second height different from the first height.

Clause 27: The article of clause 26, where the first subplurality of microscale protrusions is interleaved with the second subplurality of microscale protrusions.

Clause 28: The article of any of clauses 1 to 27, where each microscale protrusion of the plurality of microscale protrusions is identical to at least one other protrusion of the plurality of microscale protrusions in at least one of size or shape.

Clause 29: The article of clause 28, where each microscale protrusion of the plurality of microscale protrusions is identical to at least one other protrusion of the plurality of microscale protrusions in both size and shape.

Clause 30: The article of any of clauses 1 to 29, where the plurality of microscale protrusions is equally spaced along the antimicrobial region.

Clause 31: The article of any of clauses 1 to 30, where at least a first pair of microscale protrusions of the plurality of microscale protrusions defines a first minimum inter-protrusion distance, and where at least a second pair of microscale protrusions of the plurality of microscale protrusions defines a second minimum inter-protrusion distance different from the first minimum inter-protrusion distance.

Clause 32: The article of any of clauses 1 to 31, where the plurality of microscale protrusions includes a plurality of clusters, each cluster of the plurality of clusters defining a respective intra-cluster minimum distance different from a respective minimum inter-cluster distance.

Clause 33: The article of clause 32, where the respective intra-cluster minimum distance is less than the respective minimum inter-cluster distance.

Clause 34: The article of any of clauses 1 to 33, where at least one of a length, a width, or a height of each microscale protrusion of the plurality of microscale protrusions is less than or equal to 500 μm.

Clause 35: The article of clause 34, where a height of each microscale protrusion of the plurality of microscale protrusions is in a range of from 1 μm to 500 μm.

Clause 36: The article of clauses 34 or 35, where a width of each microscale protrusion of the plurality of microscale protrusions is in a range of from 50 μm to 350 μm.

Clause 37: The article of any of clauses 34 to 36, where a maximum diameter of each microscale protrusion of the plurality of microscale protrusions is in a range of from 10 μm to 50 μm.

Clause 38: The article of any of clauses 34 to 37, where a length of each microscale protrusion of the plurality of microscale protrusions is in a range of from 50 μm to 350 μm.

Clause 39: The article of any of clauses 34 to 38, where a contour radius of each microscale protrusion of the plurality of microscale protrusions is in a range of from 5 μm to 20 μm.

Clause 40: The article of any of clauses 1 to 39, where the plurality of microscale protrusions defines an average inter-protrusion spacing in a range of from 10 μm to 300 μm.

Clause 41: The article of any of clauses 1 to 40, where each microscale protrusion of the plurality of microscale protrusions includes at least one polymer.

Clause 42: The article of clause 41, where each microscale protrusion of the plurality of microscale protrusions consists essentially of the at least one polymer.

Clause 43: The article of clauses 41 or 42, where the at least one polymer includes at least one of a cyclic olefin copolymer, a copolyester, a polypropylene, a polysulfone, a polyphthalamide, or a poly (phenylene methylene).

Clause 44: The article of any of clauses 1 to 43, where a composition of the housing is the same as a composition of the plurality of microscale protrusions.

Clause 45: The article of any of clauses 1 to 44, where the housing includes a cylinder or a semi-cylinder.

Clause 46: The article of any of clauses 1 to 45, where the housing is rounded.

Clause 47: The article of any of clauses 1 to 46, where the housing is substantially rigid.

Clause 48: The article of any of clauses 1 to 47, where the fluid for dialysis includes water.

Clause 49: The article of any of clauses 1 to 48, where at least a portion of the housing is transparent or translucent.

Clause 50: The article of any of clauses 1 to 49, wherein the housing further includes at least one antimicrobial agent.

Clause 51: The article of clause 50, wherein the at least one antimicrobial agent is dispersed in a matrix of the housing.

Clause 52: The article of clauses 50 or 51, wherein the at least one antimicrobial agent is dispersed in a matrix of the microscale texture.

Clause 53: The article of any of clauses 47 to 52, wherein an antimicrobial coating applied to at least a portion of the housing includes the at least one antimicrobial agent.

Clause 54: The article of clause 53, wherein the antimicrobial coating is applied over at least a portion of the at least one antimicrobial region.

Clause 55: The article of clauses 53 or 54, wherein the antimicrobial coating is applied over at an entirety of the at least one antimicrobial region.

Clause 56: The article of any of clauses 50 to 55, wherein the at least one antimicrobial agent is configured to release one or both of silver ions or copper ions.

Clause 57: The article of any of clauses 1 to 56, wherein the at least one antimicrobial region has a surface roughness (Rₐ) of 0.3 or less.

Clause 58: The article of clause 57, wherein the surface roughness is 0.1 or less.

Clause 59: A system including: a dialysis container including the article of any of clauses 1 to 58; and a dialysis unit coupled to the dialysis container.

Clause 60: The system of clause 55, where the system does not include a recirculation unit configured to recirculate fluid through the housing of the article.

Clause 61: The system of clauses 59 or 60, where the system does not include a disinfection source or a disinfection line configured to disinfect the interior surface of the housing.

Clause 62: The system of any of clauses 59 to 61, where the dialysis unit is a peritoneal dialysis unit.

Clause 63: A method including forming the article of any of clauses 1 to 58.

Clause 64: The method of clause 63, where the forming the article includes fabricating the microscale texture by at least one of photolithography, electron beam lithography, soft lithography, x-ray lithography, plasma etching, reactive ion etching, chemical vapor deposition, layer-by-layer deposition, plasma-enhanced chemical vapor deposition, micro-molding, or additive manufacturing.

Various examples have been described. These and other examples are within the scope of the following claims.

What is claimed is:

1. An article comprising:
a housing configured to receive a fluid for dialysis,
wherein the housing defines an interior surface configured to contact the fluid when the fluid is received in the housing,
wherein the interior surface comprises at least one antimicrobial region,
wherein the at least one antimicrobial region defines a microscale texture configured to resist adhesion of bacteria and resist formation of biofilm, and
wherein the microscale texture comprises a plurality of microscale protrusions extending from the interior surface.

2. The article of claim 1, wherein at least one microscale protrusion of the plurality of microscale protrusions is solid.

3. The article of claim 1, wherein at least one microscale protrusion of the plurality of microscale protrusions defines a hollow interior.

4. The article of claim 1, wherein at least one microscale protrusion of the plurality of microscale protrusions is integral with the housing.

5. The article of claim 1, wherein the plurality of microscale protrusions comprises at least one of a pillar, a bulge, a cruciform, a star, or a polyhedron.

6. The article of claim 5, wherein the plurality of microscale protrusions comprises a plurality of pillars comprising the pillar, and wherein the pillar defines a circular contour or an elliptical contour.

7. The article of claim 6, wherein the plurality of microscale protrusions comprises a plurality of stars comprising the star, and wherein the plurality of stars is interleaved with the plurality of pillars.

8. The article of claim 7, wherein the plurality of microscale protrusions comprises a plurality of polyhedrons comprising the polyhedron, and wherein the plurality of polyhedrons is interleaved with the plurality of pillars and the plurality of stars.

9. The article of claim 5, wherein the plurality of microscale protrusions comprises a plurality of cruciforms comprising the cruciform, and
wherein the plurality of cruciforms is interleaved with one another.

10. The article of claim 9, wherein each cruciform of the plurality of cruciforms has a height of 20 μm, a width of 23 μm, and an inter-cruciform spacing of 5 μm.

11. The article of claim 1, wherein each microscale protrusion of the plurality of microscale protrusions defines a same height.

12. The article of claim 1, wherein the plurality of microscale protrusions comprises a plurality of clusters, each cluster of the plurality of clusters defining a respective intra-cluster minimum distance different from a respective minimum inter-cluster distance.

13. The article of claim 1, wherein at least one of a length, a width, or a height of each microscale protrusion of the plurality of microscale protrusions is less than or equal to 500 μm.

14. The article of claim 1, wherein the housing comprises at least one polymer, the at least one polymer comprising at least one of a cyclic olefin copolymer, a copolyester, a polypropylene, a polysulfone, a polyphthalamide, or a poly (phenylene methylene).

15. The article of claim 14, wherein the housing further comprises at least one antimicrobial agent.

16. The article of claim 15, wherein the at least one antimicrobial agent is dispersed in a matrix of the housing or in an antimicrobial coating applied to at least a portion of the housing.

17. The article of claim 1, wherein the at least one antimicrobial region has a surface roughness (Rₐ) of 0.3 or less.

18. A system comprising:
a dialysis container comprising an article comprising a housing configured to receive a fluid for dialysis; and
a dialysis unit coupled to the dialysis container,
wherein the housing defines an interior surface configured to contact the fluid when the fluid is received in the housing,
wherein the interior surface comprises at least one antimicrobial region,
wherein the at least one antimicrobial region defines a microscale texture configured to resist adhesion of bacteria and resist formation of biofilm, and
wherein the microscale texture comprises a plurality of microscale protrusions extending from the interior surface.

19. The system of claim 18, wherein at least one of a length, a width, or a height of each microscale protrusion of the plurality of microscale protrusions is less than or equal to 500 μm.

20. A method comprising forming an article comprising a housing configured to receive a fluid for dialysis,
wherein the housing defines an interior surface configured to contact the fluid when the fluid is received in the housing,
wherein the interior surface comprises at least one antimicrobial region,
wherein the at least one antimicrobial region defines a microscale texture configured to resist adhesion of bacteria and resist formation of biofilm, and
wherein the microscale texture comprises a plurality of microscale protrusions extending from the interior surface.
